(19) United States
(12) Patent Application Publication
(10) Pub. No.: US 2015/0164627 A1
(43) Pub. Date: Jun. 18, 2015

(54) MEDICAL TUBE AND MEDICAL TUBE ASSEMBLY

(71) Applicants: Masakatsu KAWAURA, Sunnyvale, CA (US); Nao YOKOI, Sunnyvale, CA (US)

(72) Inventors: Masakatsu KAWAURA, Sunnyvale, CA (US); Nao YOKOI, Sunnyvale, CA (US)

(73) Assignee: TERUMO KABUSHIKI KAISHA, Tokyo (JP)

(21) Appl. No.: 14/630,779

(22) Filed: Feb. 25, 2015

Related U.S. Application Data
(63) Continuation of application No. PCT/JP2012/071596, filed on Aug. 27, 2012.

(51) Int. Cl. A61F 2/00; A61B 17/34

(52) U.S. Cl. CPC A61F 2/0045; A61B 17/3468

(57) ABSTRACT

A medical tube and a medical tube assembly are disclosed which can enable an implant to be relatively easily and reliably inserted and placed to indwell in a living body. The medical tube can accept an elongated implant inserted in the medical tube. The medical tube can be composed of a tube which has a distal end opening where its distal end is open, and a proximal end opening where its proximal end is open. The tube has a curved section where an intermediate portion in the longitudinal direction of the medical tube is curved and where the curved state is maintained.

### CROSS-REFERENCES TO RELATED APPLICATIONS

This application claims priority to International Application No. PCT/JP2012/071596 filed on Aug. 27, 2012, the entire content of which is incorporated herein by reference.

### TECHNICAL FIELD

The present disclosure generally relates to a medical tube and a medical tube assembly.

### BACKGROUND DISCUSSION

In a patent suffering from urinary incontinence, particularly stress urinary incontinence, urine leakage can occur due to an abdominal pressure exerted during a normal exercise or by laughing, coughing, sneezing or the like. This can be attributable, for example, to loosening of the pelvic floor muscle, which is a muscle for supporting the urethra, caused by childbirth or the like.

For treatment of urinary incontinence, effective is surgical therapy, in which there is used, for example, a tape-shaped implant called a “sling,” and the sling is placed indwelling in the body, so as to support the urethra thereby (see, for example, Japanese Patent Laid-Open No. 2010-99499). In order to put a sling indwelling in the body, an operator incises the vagina with a surgical knife, exfoliates a biological tissue between the urethra and the vagina, and forms a puncture-through hole for providing communication between the exfoliated biological tissue and the exterior, by use of a puncture needle. Then, the sling can be inserted into the puncture-through hole, to be placed to indwell in the exfoliated biological tissue in the body.

Meanwhile, at the time of inserting the sling into the puncture-through hole, the inserting operation is conducted while the sling is kept inserted in a flexible tube. Since this tube is flexible, it would be crushed (compressed) by the exfoliated biological tissue. As a result, if the sling inserting operation is conducted, the sling-inserting operation may become relatively difficult to carry out, due to the friction between the tube and the biological tissue.

### SUMMARY

In accordance with an exemplary embodiment, a medical tube and a medical tube assembly is disclosed, which can help ensure that an operation of inserting an implant into a living body so as to set the implant to indwell in the living body can be carried out relatively easily and reliably.

In accordance with an exemplary embodiment, a medical tube in which an elongated implant is to be inserted, can include a tube having a distal end opening where a distal end of the distal end opening is open, and a proximal end opening where a proximal end of the proximal end opening is open, the tube having a curved section where an intermediate portion in a longitudinal direction of the medical tube is curved and where a curved state is maintained.

In accordance with an exemplary embodiment, the tube can be rigid at least at the curved section of the tube.

In accordance with an exemplary embodiment, the curved section can be curved in a circular arc shape.

In accordance with an exemplary embodiment, the medical tube can include a separation part where the tube is separable at an intermediate portion in the longitudinal direction of the medical tube.

In accordance with an exemplary embodiment, the separation part can be located at a central portion in a longitudinal direction of the curved section.

In accordance with an exemplary embodiment, the tube can include a marker for grasping of the central portion in the longitudinal direction of the curved section.

In accordance with an exemplary embodiment, the tube is separated at the separation part into a first tube on a distal side and a second tube on a proximal side, and the separation part is a part having a fitting structure in which a distal portion of the second tube is fitted in a proximal portion of the first tube, before separation of the tube into the first tube and the second tube.

In accordance with an exemplary embodiment, the medical tube can be circular in cross-sectional shape at least near the distal end opening.

In accordance with an exemplary embodiment, an enlarged diameter portion where the medical tube is enlarged in outside diameter is provided near the distal end opening.

In accordance with an exemplary embodiment, the medical tube can be flat in cross-sectional shape at its portion proximally of its portion near the distal end opening.

In accordance with an exemplary embodiment, the width of the flat shape is equal to or greater than a maximum outside diameter near the distal end opening of the medical tube.

In accordance with an exemplary embodiment, a thickness direction of the flat shape of the medical tube is oriented toward a center-of-curvature of the curved section.

In accordance with an exemplary embodiment, the medical tube includes at least one lumen which opens respectively at the distal end opening and at the proximal end opening, wherein the implant is inserted in the lumen.

In accordance with an exemplary embodiment, the medical tube includes a plurality of the lumens, wherein a guide wire is inserted in each of other lumen or lumens than that one of the lumens in which the implant is inserted.

In accordance with an exemplary embodiment, the medical tube assembly includes the medical tube as disclosed herein and an elongated implant, which can be inserted in the medical tube.

In accordance with an exemplary embodiment, at the time of inserting the medical tube into a living body, the curved section can be prevented from being crushed (compressed) inside the living body, which can help ensure that, for example, in the case where an implant is preliminarily inserted in the medical tube, an operation of inserting the medical tube into a living body together with the implant can be carried out relatively easily and assuredly.

In addition, after the inserting operation, the medical tube can be pulled out of the living body, and whereby the implant is left as it is, so that the implant is relatively easily and reliably placed to indwell in the living body.

In addition, where the medical tube has the separation part, the medical tube can be separated at the separation part, such that the operation of withdrawing the medical tube out of a living body can be carried out relative ease, and, the implant indwelling can be placed with relatively speedily in the living body.

In accordance with an exemplary embodiment, a method is disclosed of placing an implant in living body tissue, the method comprising: forming a puncture hole by puncturing a living body tissue with a puncture apparatus, the puncture apparatus including a puncture needle and an outer tube; withdrawing the puncture needle of the puncture member from the outer tube; inserting a guide wire inserted into and passing through the outer tube, such that a distal portion of the guide wire protrudes from an opening on a distal end of the outer tube and a proximal portion of the guide wire protrudes from an opening on a proximal end of the outer tube; withdrawing the outer tube from the puncture hole, while leaving the guide wire extending through the puncture hole; inserting a medical tube assembly into the puncture hole, the medical tube assembly including a medical tube and an elongated implant within the medical tube, the medical tube having a distal end opening where a distal end of the distal end opening is open, and a proximal end opening where a proximal end of the proximal end opening is open, and wherein the medical tube includes a curved section where an intermediate portion in a longitudinal direction of the tube is curved and where a curved state is maintained; pulling the guide wire out of the medical tube assembly; and withdrawing the medical tube from the puncture hole and leaving the implant in a state in a state where a distal-side portion of the implant protrudes from a body surface on one side and a proximal-side portion of the implant protrudes from the body surface on another side.

### BRIEF DESCRIPTION OF DRAWINGS

FIG. 1 is a sectional view for sequentially illustrating a method of using the medical tube (medical tube assembly) of the present disclosure in a first exemplary embodiment.

FIG. 2 is a sectional view for sequentially illustrating the method of using the medical tube (medical tube assembly) of the present disclosure in the first exemplary embodiment.

FIG. 3 is a sectional view for sequentially illustrating the method of using the medical tube (medical tube assembly) of the present disclosure in the first exemplary embodiment.

FIG. 4 is a sectional view for sequentially illustrating the method of using the medical tube (medical tube assembly) of the present disclosure in the first exemplary embodiment.

FIG. 5 is a sectional view for sequentially illustrating the method of using the medical tube (medical tube assembly) of the present disclosure in the first exemplary embodiment.

FIG. 6 is a sectional view for sequentially illustrating the method of using the medical tube (medical tube assembly) of the present disclosure in the first exemplary embodiment.

FIG. 7 is a sectional view for sequentially illustrating the method of using the medical tube (medical tube assembly) of the present disclosure in the first exemplary embodiment.

FIG. 8 is a sectional view for sequentially illustrating the method of using the medical tube (medical tube assembly) of the present disclosure in the first exemplary embodiment.

FIG. 9 is a sectional view for sequentially illustrating the method of using the medical tube (medical tube assembly) of the present disclosure in the first exemplary embodiment.

FIG. 10 is a sectional view for sequentially illustrating the method of using the medical tube (medical tube assembly) of the present disclosure in the first exemplary embodiment.

FIG. 11 is a sectional view for sequentially illustrating the method of using the medical tube (medical tube assembly) of the present disclosure in the first exemplary embodiment.

FIG. 12 is a side view taken in the direction of arrow 12 in FIG. 1.

FIG. 13 is a sectional view taken along line 13-13 in FIG. 7.

FIG. 14 is a sectional view taken along line 14-14 in FIG. 7.

FIGS. 15A and 15B show views taken in the direction of arrow 15 in FIG. 7, where FIG. 15A shows a state before separation of a separation part, and FIG. 15B shows a state after separation of the separation part.

FIG. 16 is a cross-sectional view showing a second exemplary embodiment of the medical tube (medical tube assembly) of the present disclosure.

FIG. 17 is a sectional view for sequentially illustrating a method of using the medical tube (medical tube assembly) of the present disclosure in a third exemplary embodiment.

FIG. 18 is a sectional view for sequentially illustrating the method of using the medical tube (medical tube assembly) of the present disclosure in the third exemplary embodiment.

FIG. 19 is a sectional view for sequentially illustrating the method of using the medical tube (medical tube assembly) of the present disclosure in the third exemplary embodiment.

### DETAILED DESCRIPTION

Hereafter, the medical tube and the medical tube assembly of the present disclosure will be described in detail based on preferred embodiments of the disclosure shown in the attached drawings.

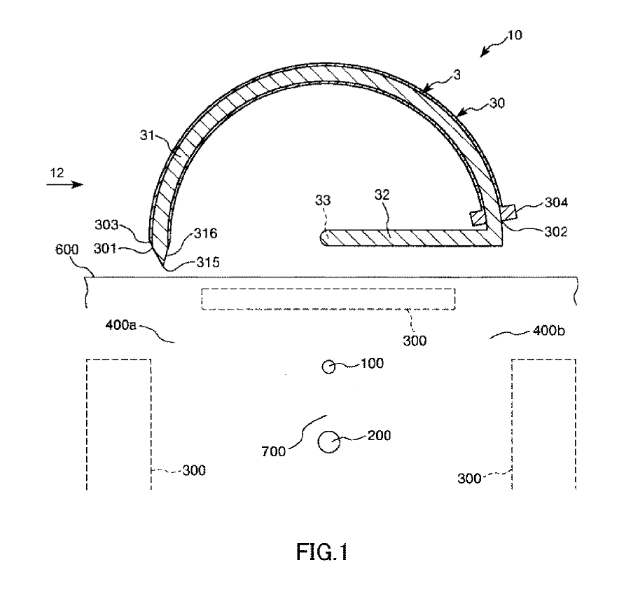

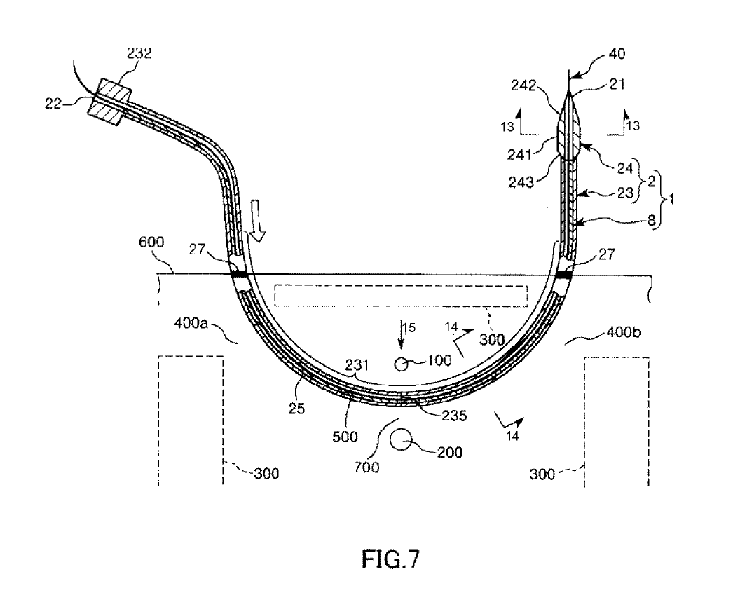

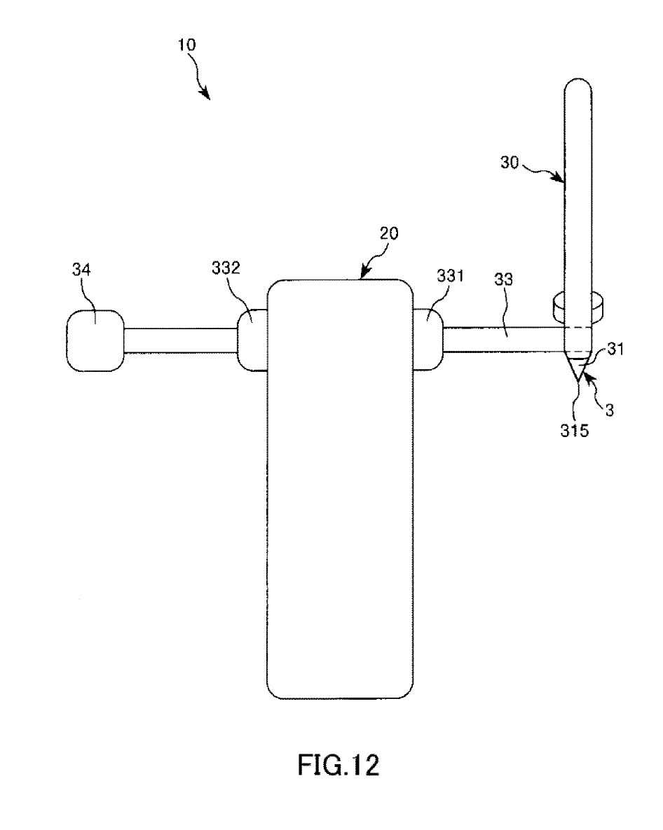

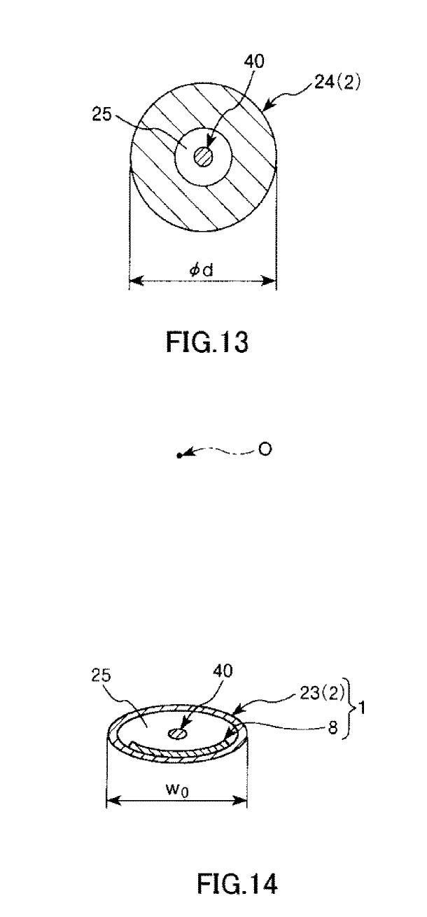

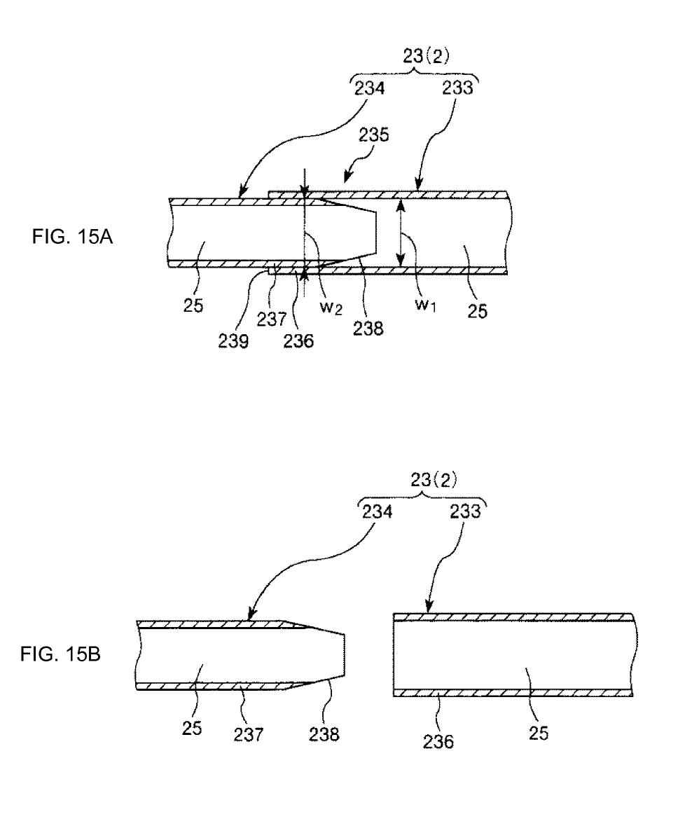

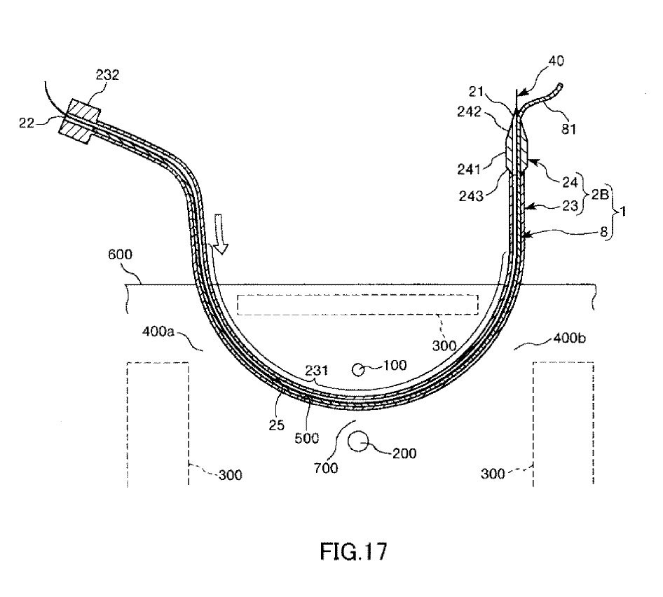

FIGS. 1 to 11 are sectional views for sequentially illustrating a method of using the medical tube (medical tube assembly) of the present disclosure in a first exemplary embodiment; FIG. 12 is a view (side view) taken in the direction of arrow 12 in FIG. 1; FIG. 13 is a sectional view taken along line 13-13 in FIG. 7; FIG. 14 is a sectional view taken along line 14-14 in FIG. 7; and FIGS. 15A and 15B show views taken along the direction of arrow 15 in FIG. 7, where FIG. 15A shows a state before separation of a separation part, and FIG. 15B shows a state after separation of the separation part). Hereafter, for convenience of description, the upper side in FIGS. 1 to 12 (and in FIGS. 17 to 19, as well) will be referred to as “upper (side)” or “above,” and the lower side as “lower (side)” or “below.” In addition, the side of a needle tip will be referred to as “distal end,” and the opposite side as “proximal end.”

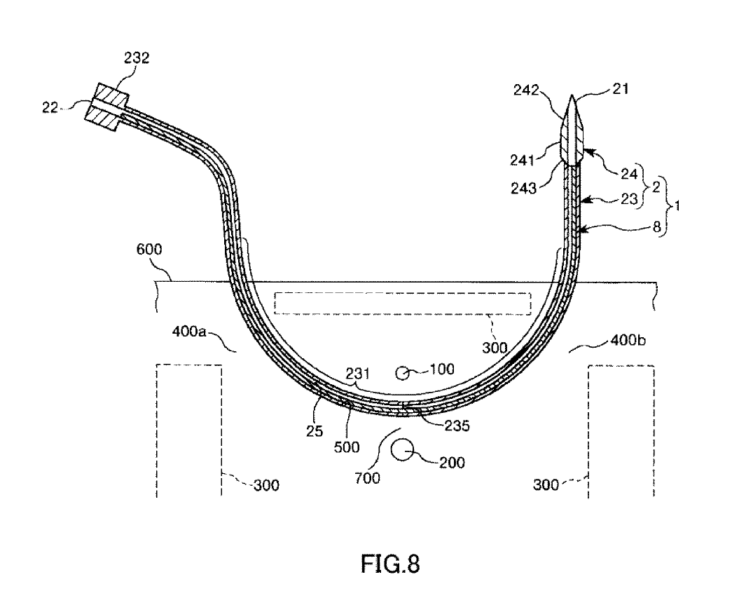

A medical tube assembly 1 as shown in FIGS. 7 and 8, can include a medical tube (tube) 2, and an implant 8 which is inserted in the medical tube 2. In accordance with an exemplary embodiment, the medical tube assembly 1 can be a medical instrument that can be used for treatment of female urinary incontinence. Now, the configurations of components will be described.

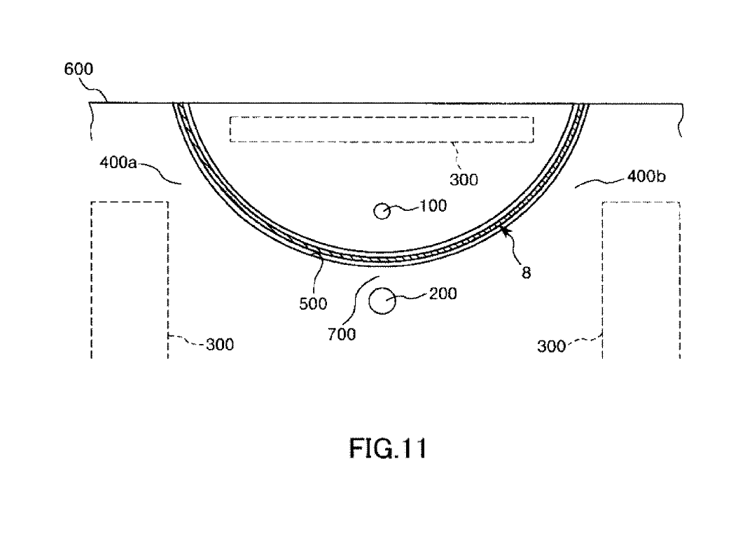

The implant 8, which is generally called a “sling,” is an implantable instrument for treatment of female urinary incontinence, for example, an instrument for supporting a urethra 100, specifically, an instrument which supports the urethra 100 in the manner of pulling the urethra 100 in a direction for spacing away from a vagina 200 when the urethra 100 would tend to move toward the vagina 200 side, for example (see FIG. 11). The implant 8 can be composed of a member, which is flexible and band-like (elongated) in shape (see FIGS. 7 to 11).

The material constituting the implant 8 is not specifically restricted, for example, various resin materials that are biocompatible can be used as the material.

The implant 8 may be preliminarily inserted (housed) in the medical tube 2 as illustrated in FIG. 8, or may be inserted into the medical tube 2 in the course of a procedure. Where the implant 8 is preliminarily inserted in the medical tube 2, a relatively speedy procedure can be performed. Where the implant 8 is inserted into the medical tube 2 in the course of a procedure, an implant 8 suited to the individual case can be selected each time, according to the case. In this embodiment, a case where the implant 8 is preliminarily inserted in the medical tube 2 will be described on a representative basis.

In the next place, prior to describing the medical tube 2, a puncture apparatus 10 to be used together with the medical tube assembly 1 in treatment of female urinary incontinence will be described. In this exemplary embodiment, a “medical instrument set” for treatment of female urinary incontinence can be composed of the puncture apparatus 10 and the medical tube assembly 1.

As shown in FIGS. 1 to 5 and 12, the puncture apparatus 10 can include a puncture member 3, a support member 20 supporting the puncture member 3 in a rotatable manner, and an outer tube 30 in which to insert the puncture member 3. The puncture apparatus 10 may further include a bar-shaped urethral-insertion member (not shown) to be inserted into the urethra 100, and a bar-shaped vaginal-insertion member (not shown) to be inserted into the vagina 200. The bar-shaped urethral-insertion member and the bar-shaped vaginal-insertion members are preferably supported by and fixed to the support member 20.

The puncture member 3 can include a puncture needle 31 for puncturing a biological tissue 700, a shaft portion 33, and an interlock portion 32 that interlocks the puncture needle 31 and the shaft portion 33.

The puncture needle 31 can have a sharp needle tip 315 at the distal end of the puncture needle 31, and can be curved in a circular arc shape with a center on the shaft portion 33. In addition, the axis of the puncture needle 31 and the axis of the shaft portion 33 are in a skew-lines relationship, which can help ensure that when the puncture member 3 is rotationally moved about the shaft portion 33, the needle tip 315 of the puncture needle 31 is moved along the circular arc, in a plane orthogonal to the axis of the shaft portion 33, for example, in a plane such that the axis of the shaft portion 33 constitutes a normal to the plane.

The center angle of the circular arc of the puncture needle 31 is not particularly limited but is set, as necessary, according to various conditions. For example, the center angle can be set that when the biological tissue 700 is punctured by the puncture needle 31, a puncture-through hole (puncture hole) 500 having a circular arc shape is formed in the biological tissue 700, as will be described later. Such a center angle is, for example, preferably 120 to 270 degrees, more preferably 160 to 230 degrees, and further preferably 180 to 210 degrees.

In addition, while the needle tip 315 of the puncture needle 31 is oriented counterclockwise in FIGS. 1 to 3 in this embodiment, this is not restrictive, and the needle tip 315 may be oriented clockwise.

In accordance with an exemplary embodiment, the puncture needle 31 can be formed with a tapered portion 316 where its outside diameter gradually increases along the proximal direction from the needle tip 315.

In addition, the puncture needle 31 may be either a solid needle or a hollow needle.

In accordance with an exemplary embodiment, the shaft portion 33 can serve as a rotating shaft of the puncture member 3 (puncture needle 31), and can be rotatably disposed on the support member 20.

As shown in FIG. 12, the shaft portion 33 penetrates the support member 20 in the left-right direction in the figure. In addition, a flange 331 and a flange 332 are formed respectively at a distal-side portion and a proximal-side portion of the shaft portion 33, with the support member 20 interposed between the flange 331 and the flange 332. The flanges 331 and 332 restrict movement of the shaft portion 33 in the axial direction relative to the support member 20.

In addition, at an end portion of the shaft portion 33 on the side opposite to the puncture needle 31, a grasping unit 34 can be provided as an operation unit for rotationally operating the puncture member 3. The grasping unit 34 can be in the shape of a rectangular parallelepiped in this exemplary embodiment. At the time of rotationally moving the puncture member 3, the grasping unit 34 is grasped with fingers and is rotated in a predetermined direction. The shape of the grasping unit 34 is not restricted to the disclosed shape.

In accordance with an exemplary embodiment, the interlock portion 32 is a portion that interlocks the proximal end of the puncture needle 31 and the shaft portion 33.

The material constituting the puncture member 3 is not specifically restricted; for example, various metallic materials such as stainless steels, aluminum or aluminum alloys, titanium or titanium alloys, etc. can be used as the material.

In accordance with an exemplary embodiment, the support member 20 is a member that supports the puncture member 3 in a rotatable manner. The support member 20 is omitted in FIGS. 1 to 3.

The support member 20 can restrict the position of the puncture member 3 in such a manner that the needle tip 315 of the puncture needle 31 passes between the urethra 100 and the vagina 200 when the puncture member 3 is rotationally moved to puncture the biological tissue 700, which can help ensure that the puncture-through hole 500 having a circular arc shape is formed by the puncture needle 31, between the urethra 100 and the vagina 200.

The material constituting the support member 20 is not particularly limited; for example, various resin materials such as polyethylene, polypropylene, etc. can be used as the material.

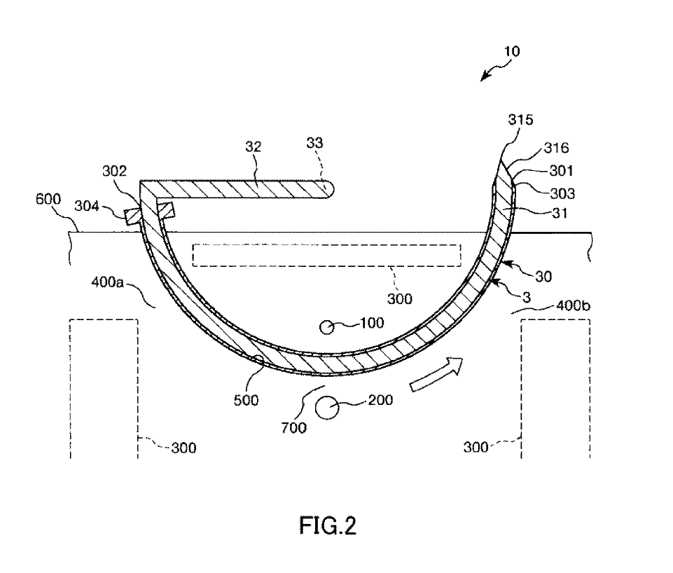

As shown in FIGS. 1 and 2, the outer tube 30 is a member which is preliminarily mounted to the puncture needle 31 of the puncture member 3, for example, a member in which the puncture needle 31 of the puncture member 3 is preliminarily inserted. In accordance with an exemplary embodiment, the outer tube 30 is preferably a rigid tube. In accordance with an exemplary embodiment, the outer tube 30 can be curved in a circular arc shape, to the same extent as the puncture needle 31. In accordance with an exemplary embodiment, the term “rigid” can refer to a degree of rigidity such that the outer tube 30 can, by itself, maintain the curved state in the circular arc shape.

The outer tube 30 has a distal end opening 301 where its distal end is open, and a proximal end opening 302 where its proximal end is open.

In addition, a distal portion of the outer tube 30 can be formed at its outer circumferential portion with a tapered portion 303 which can be equal in taper angle to the tapered portion 316 of the puncture needle 31. In an assembled state wherein the outer tube 30 is mounted to the puncture needle 31 to achieve assembly, the tapered portion 316 of the puncture needle 31 and the tapered portion 303 of the outer tube 30 constitute a single continuous tapered section, which can help ensure that in the assembled state, the outer tube 30 is, together with the puncture needle 31, able to puncture the biological tissue 700 and, therefore, to form the puncture-through hole 500 in the biological tissue 700 (see FIG. 2).

In accordance with an exemplary embodiment, a proximal portion of the outer tube 30 is formed at its outer circumferential portion with a flange portion 304 where its outside diameter is enlarged. Where the flange portion 304 is attached to a living body surface 600 in a state as shown in FIG. 2, for example, a limit on rotary movement in the distal direction of the puncture needle 31 is restricted, so that the biological tissue 700 can be punctured neither too much nor too little.

The material constituting the outer tube 30 is not specifically restricted; for example, various resin materials such as polyethylene, polypropylene, etc. and various metallic materials such as stainless steels, aluminum or aluminum alloys, titanium or titanium alloys, etc. can be used as the material.

In accordance with an exemplary embodiment as shown in FIGS. 7 to 9, the medical tube 2 is a tube which has a distal end opening (distal end) 21 where its distal end is open, and a proximal end opening (proximal end) 22 where its proximal end is open. In addition, the medical tube 2 can be sectioned into an elongated tube main body 23, and a head part 24 provided at a distal portion of the tube main body 23. In accordance with an exemplary embodiment, the medical tube 2 can be formed with a lumen 25, which can penetrate the tube main body 23 and the head part 24, for example, which opens respectively at the distal end opening 21 and at the proximal end opening 22. In accordance with an exemplary embodiment, the implant 8 can be inserted into the lumen 25.

As shown in FIGS. 7 and 8, the tube main body 23 can have a curved section 231 where an intermediate portion in the longitudinal direction of the tube main body 23 can be curved in a circular arc shape. In accordance with an exemplary embodiment, at least the curved section 231 of the medical tube 2 can be rigid. Here, the term “rigid” refers to a degree of rigidity such that the curved section 231 can, by itself, maintain the curved state in the circular arc shape. In addition, the degree of curling (curvature) of the curved section 231 is substantially the same as that of the puncture needle 31 of the puncture apparatus 10.

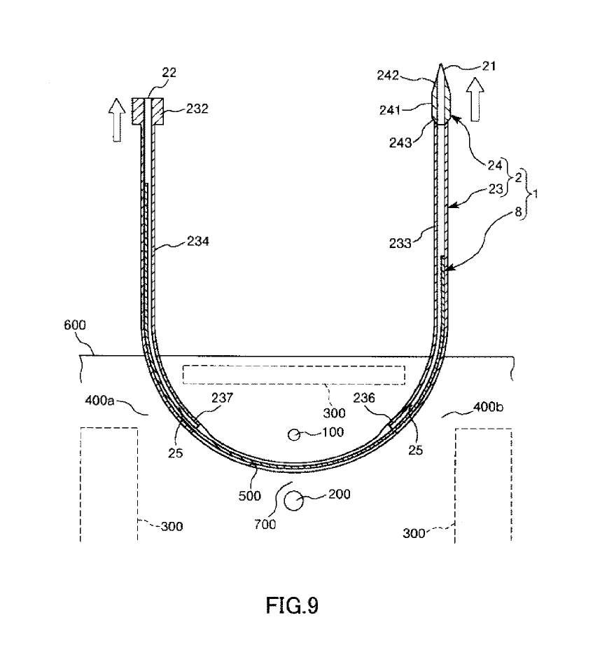

Such a configuration can help ensure that when the medical tube 2 is inserted into the puncture-through hole 500 formed by the puncture apparatus 10, the curved section 231 can be prevented from being crushed (compressed) within the puncture-through hole 500, and the curved section 231 can relatively easily be shaped following (along) the curved shape of the puncture-through hole 500, which can help enable the operation of inserting the medical tube 2 into the puncture-through hole 500 (living body) together with the implant 8 to be carried out relatively easily and reliably. In addition, by separating the medical tube 2 as will be described later, after this inserting operation, the implant 8 can be relatively easily and assuredly be placed to indwell in the puncture-through hole 500 (see FIG. 9).

In accordance with an exemplary embodiment, the portion other than the curved section 231 of the tube main body 23 may be rigid or may be non-rigid, for example, flexible.

As shown in FIG. 14, the cross-sectional shape of the tube main body 23, for example, the cross-sectional shape of that portion of the medical tube 2 which is on the proximal side of the head part 24 (the vicinity of the distal end opening 21), can be a flat shape, for example, an elliptic shape, which can help ensure that at the time of inserting the band-shaped implant 8 into the lumen 25 preliminarily, the inserting operation can be carried out relatively easily. In addition, there is a merit that a space allowing reliable insertion of the implant 8 can be formed inside the puncture-through hole 500, and, further, the orientation of the implant 8 can be restricted.

The cross-sectional shape of the tube main body 23 may be other than the flat shape, for example, a circular shape.

In accordance with an exemplary embodiment, as shown in FIG. 14, the thickness direction (minor-diameter direction) of the flat shape can be oriented towards the center-of-curvature O of the curved section 231. This configuration can contribute to easier insertion of the medical tube 2 into the puncture-through hole 500, as compared with the case where the width direction (major-diameter direction) of the flat shape is oriented toward the center-of-curvature O.

As shown in FIGS. 7 to 9, a proximal portion of the tube main body 23 can be formed with a flange portion 232 where its outside diameter is enlarged. In accordance with an exemplary embodiment, the flange portion 232 may be omitted.

As shown in FIGS. 9 and 15(b), the tube main body 23 (medical tube 2) can be configured such that the tube main body 23 can be separated at an intermediate part in the longitudinal direction of the tube main body 23, and that the tube main body 23 is thereby separated into a first tube 233 on the distal side and a second tube 234 on the proximal side. In accordance with an exemplary embodiment, the separation can allow the medical tube 2 to be withdrawn relatively swiftly from the puncture-through hole 500, and, thus, only the implant 8 is placed indwelling in the puncture-through hole 500.

As shown in FIGS. 7 and 8, this separation part 235 can be disposed at a central portion in the longitudinal direction of the curved section 231, which can help ensures that when the tube main body 23 is separated at the separation part 235, as shown in FIG. 9, the urethra 100 can be suitably supported by the implant 8.

The tube main body 23 is preferably provided, respectively on its distal portion and its proximal portion, with markers 27 for grasping of the central portion in the longitudinal direction of the curved section 231 (see FIG. 7). The markers 27 can help enable reliable grasping of the position of the central portion in the longitudinal direction of the curved section 231, for example, the position of the separation part 235. While the markers 27 are provided on both the distal portion and the proximal portion of the tube main body 23 in this embodiment, this configuration is not restrictive, for example, a marker may be provided on one of the distal portion and the proximal portion of the tube main body 23.

As shown in FIG. 15A, the separation part 235 is a part having a fitting structure wherein a distal portion 237 of the second tube 234 is fitted inside a proximal portion 236 of the first tube 233, before separation of the tube main body 23 into the first tube 233 and the second tube 234, which can help ensure that by pulling the tube main body 23 toward both sides thereof, the fitted state of the proximal portion 236 of the first tube 233 and the distal portion 237 of the second tube 234 can be canceled assuredly. By this canceling, the tube main body 23 can be easily separated into the first tube 233 and the second tube 234 at the separation part 235.

In accordance with an exemplary embodiment, the distal portion 237 of the second tube 234 can be formed with a gradually decreasing width portion 238 where a width decreasing width portion 238 decreases gradually along the distal direction, which can help ensure that at the time of keeping the tube main body 23 in the state shown in FIG. 15(a), the distal portion 237 of the second tube 234 can be relatively easily inserted into the proximal portion 236 of the first tube 233, resulting in the fitted state of the end portions of them.

In addition, the inside width w₁ of the proximal portion 236 of the first tube 233 is equal to or slightly smaller than the outside width (maximum width) w₂ of the distal portion 237 of the second tube 234, which can help enable the distal portion 237 of the second tube 234 to be relatively easily inserted into the proximal portion 236 of the first tube 233.

As disclosed above, the fitting structure between the proximal portion 236 of the first tube 233 and the distal portion 237 of the second tube 234 is in a state wherein the proximal portion 236 is located outside whereas the distal portion 237 is located inside. In accordance with an exemplary embodiment, at the separation part 235, specifically at a boundary part between the proximal portion 236 and the distal portion 237, there is formed a stepped part 239 where the width is reduced stepwise along the proximal direction (see FIG. 15A). The configuration in which the stepped part 239 is formed in this way can be preferable, since the distal end of the medical tube 2 is inserted first into the puncture-through hole 500.

As shown in FIGS. 7 to 9 and 13, in accordance with an exemplary embodiment, the head part 24 can be composed of a tubular body, which can be circular in cross-sectional shape. In accordance with an exemplary embodiment, the head part 24 may be formed integrally with the tube main body 23, or may be composed of a separate body from the tube main body 23 and be joined to the tube main body 23.

The head part 24 can be provided, at an intermediate portion in the axial direction of the head part 24, with an enlarged diameter portion 241 where its outside diameter is enlarged. In addition, that portion of the head part 24 which is on the distal side of the enlarged diameter portion 241 is a tapered portion 242 where its outside diameter gradually decreases along the distal direction, and that portion of the head part 241 which is on the proximal side of the enlarged diameter portion 241 is also a tapered portion 243 where its outside diameter gradually decreases along the proximal direction. In accordance with an exemplary embodiment, the whole (or entire) length of the tapered portion 242 can be longer than the whole (or entire) length of the tapered portion 243.

With the head part 24 shaped in this fashion, the medical tube 2 can be relatively easily inserted into the puncture-through hole 500, starting with its head part 24.

In addition, as shown in FIGS. 13, 14A, and 14B, the outside width w₀ of the tube main body 23, which is flat shaped in cross section, is equal to or greater than the outside diameter φd of the head part 24, which can be circular in cross-sectional shape, and which can help ensure that as the medical tube 2 is inserted into the puncture-through hole 500, the puncture-through hole 500 can be dilated by the tube main body 23, for example, the biological tissue 700 can be exfoliated.

In accordance with an exemplary embodiment, the material constituting the medical tube 2 is not specifically restricted, and examples of the applicable material include polyolefins such as polyethylene, polypropylene, ethylene-vinyl acetate copolymer, modified polyolefins, polyamides (for example, nylon 6, nylon 46, nylon 66, nylon 610, nylon 612, nylon 11, nylon 12, nylon 6-12, nylon 6-66), thermoplastic polyimides, liquid crystal polymers such as aromatic polyesters, etc., polyphenylene oxide, polyphenylene sulfide, polycarbonate, polymethyl methacrylate, polyethers, polyether-ether ketone, polyether imides, polyacetal, various thermoplastic elastomers based on styrene, polyolefin, polyvinyl chloride, polyurethane, polyester, polyamide, polybutadiene, transpolyisoprene, fluoro-rubber, chlorinated polyethylene or the like, and copolymers, blends, polymer alloys and the like that contain these elastomers as main constituents. In accordance with an exemplary embodiment, the above-disclosed materials can be used either singly or as a mixture of two or more of the disclosed materials.

An example of the method of using the medical tube assembly 1 (medical instrument set) will be described below, referring to FIGS. 1 to 11.

First, as shown in FIG. 1, the puncture apparatus 10 in the assembled state in which the outer tube 30 is mounted to the puncture needle 31 to achieve assembly is mounted to the body surface 600 of a patient. The mounting position in this instance may be a position suitable for supporting the urethra 100 by the implant 8, which is to be implanted.

Next, the grasping unit 34 of the puncture apparatus 10 is grasped with one hand, and, as shown in FIG. 2, the puncture member 3 is rotated counterclockwise in the figure. As a result, the puncture needle 31, together with the outer tube 30, is moved with the shaft portion 33 as a center of rotary movement to progressively pass, or puncture, a left-side inguinal region (or a part in the vicinity thereof) of the body surface 600 of the patient, an obturator foramen 400a of a pelvis 300, a region between the urethra 100 and the vagina 200, an obturator foramen 400b of the pelvis 300, and a right-side inguinal region (or a part in the vicinity thereof) of the body surface 600. By this puncture, the puncture-through hole 500 is formed to penetrate the biological tissue 700, extending from the left-side inguinal region to the right-side inguinal region of the body surface 600.

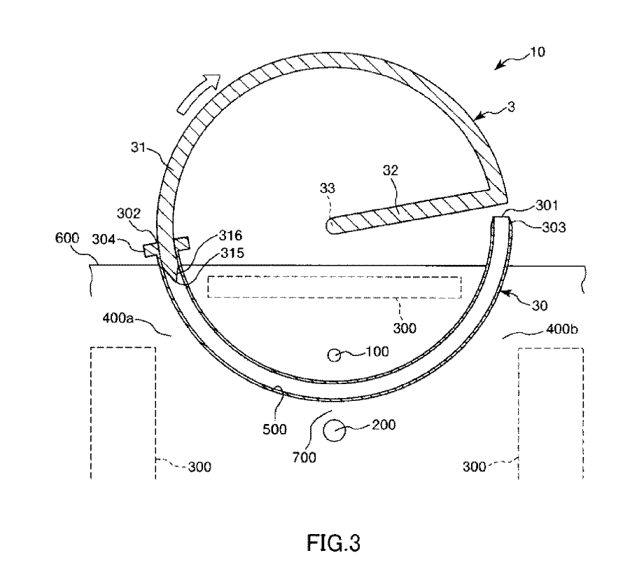

Subsequently, the grasping unit 34 of the puncture apparatus 10 is grasped with one hand, the flange portion 304 of the outer tube 30 is pressed from above by the other hand, and, as shown in FIG. 3, the puncture member 3 is rotated in a direction opposite to the aforementioned, namely, rotated clockwise in the figure. As a result, the puncture needle 31 of the puncture member 3 is drawn out of the outer tube 30. The outer tube 30 is left indwelling in the biological tissue 700.

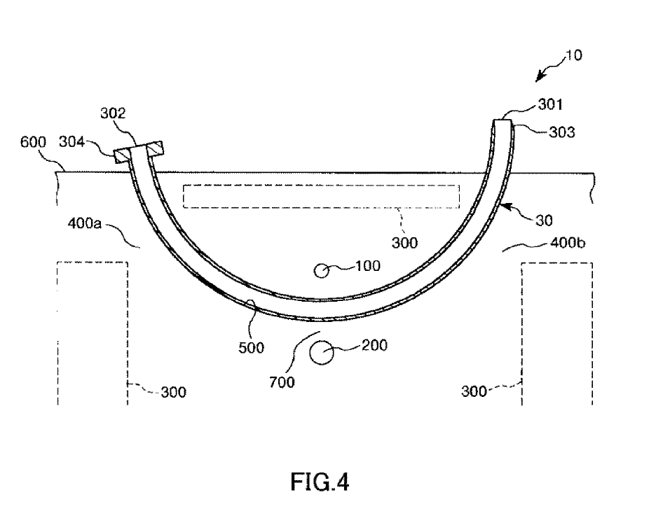

Next, as shown in FIG. 4, the puncture member 3 of the puncture apparatus 10 is removed from the body surface 600, together with the support member 20.

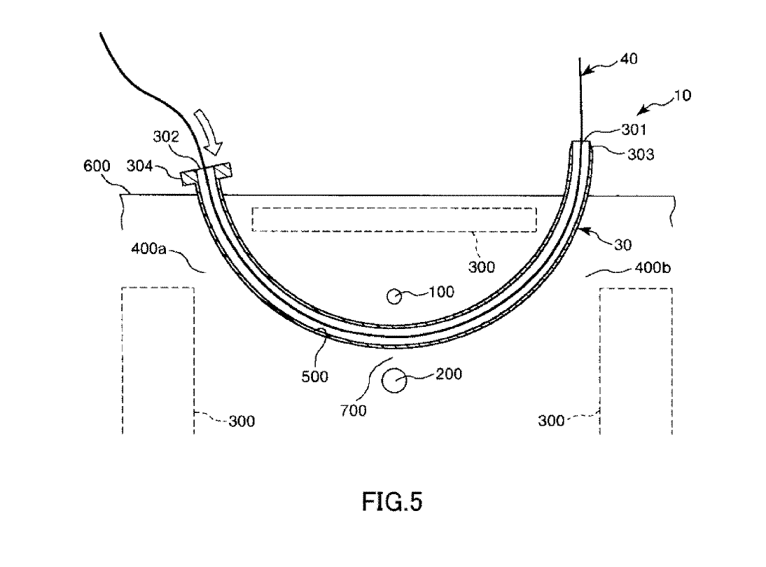

Subsequently, as shown in FIG. 5, a guide wire 40 is inserted into and passed through the outer tube 30 kept indwelling in the biological tissue 700. This results in that the guide wire 40 has its distal portion protruding from the distal end opening 301 of the outer tube 30, and has its proximal portion protruding from the proximal end opening 302 of the outer tube 30.

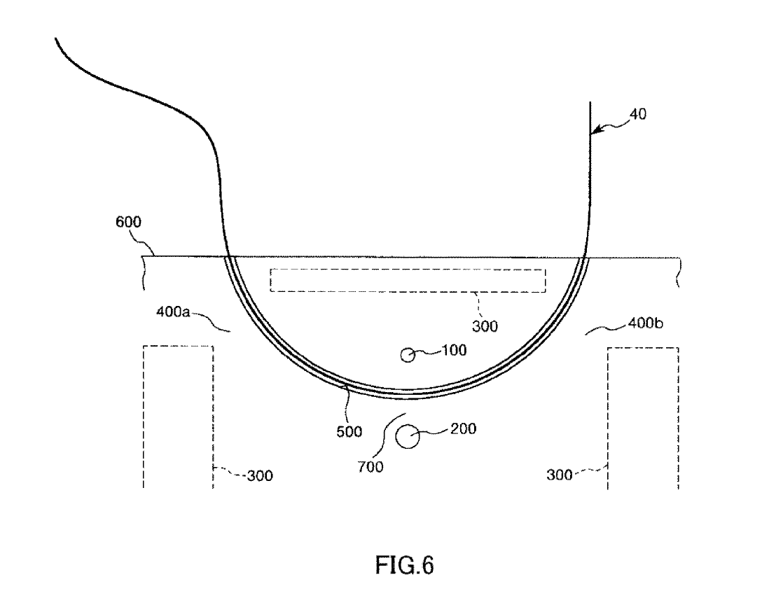

Next, a distal portion of the guide wire 40 is grasped with one hand, and, keeping this condition, the flange portion 304 of the outer tube 30 is grasped with the other hand and pulled proximally. Consequently, as shown in FIG. 6, the outer tube 30 is drawn out of the puncture-through hole 500, whereas the guide wire 40 is left extending through the puncture-through hole 500.

Subsequently, the medical tube assembly 1 in a state where the implant 8 is inserted in the medical tube 2 is prepared. Then, in a state where a proximal portion of the guide wire 40 is inserted in the distal end opening 21 of the medical tube 2, the medical tube assembly 1 is pushed distally relative to the guide wire 40, as shown in FIG. 7. This causes the medical tube assembly 1 to be inserted into and passed through the puncture-through hole 500, and brought into a state in which its distal-side portion is protruding from the body surface 600 on the side of the obturator foramen 400b, whereas its proximal-side portion is protruding from the body surface 600 on the side of the obturator foramen 400a.

Next, that portion of the medical tube assembly 1 which is protruding from the body surface 600 on the side of the obturator foramen 400b is grasped with one hand, and, keeping this condition, a proximal portion of the guide wire 40 is grasped with the other hand and pulled proximally. This results, as shown in FIG. 8, that the guide wire 40 is pulled out of the medical tube assembly 1 (medical tube 2), whereas the medical tube assembly 1 is left extending through the puncture-through hole 500.

Subsequently, the head part 24 of the medical tube 2 is grasped with one hand, the flange portion 232 is grasped with the other hand, and the head part 24 and the flange portion 232 are pulled in opposite directions, as shown in FIG. 9. This causes the medical tube 2 to be separated at the separation part 235 into the first tube 233 and the second tube 234.

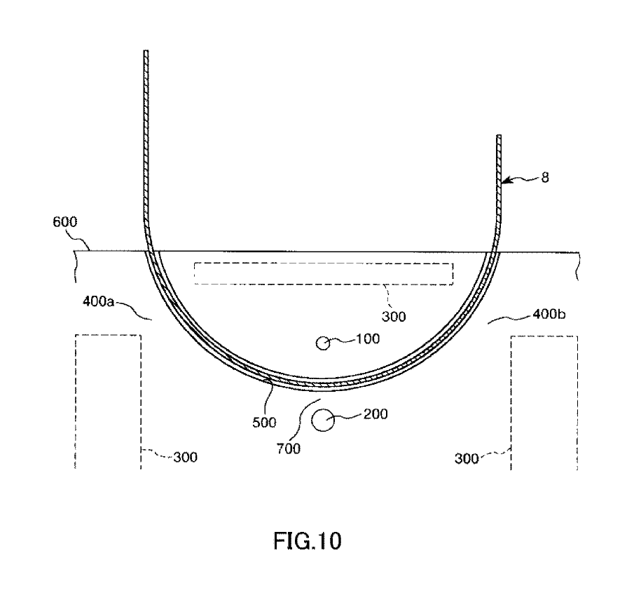

Next, the first tube 233 and the second tube 234 are respectively drawn out of the puncture-through hole 500, resulting in that the implant 8 is left extending through the puncture-through hole 500, as shown in FIG. 10. The implant 8 is in a state where its distal-side portion is protruding from the body surface 600 on the side of the obturator foramen 400b, whereas its proximal-side portion is protruding from the body surface 600 on the side of the obturator foramen 400a. Then, the distal-side portion and the proximal-side portion of the implant 8 are pulled with predetermined forces, respectively. This generates a tension on the implant 8, whereby the urethra 100 is pulled in a direction for spacing away from the vagina 200, and supported from below by the implant 8.

Subsequently, as shown in FIG. 11, unnecessary portions of the implant 8 are cut away, and predetermined wound closure and the like are carried out, to complete the procedure.

In such a procedure as above, namely, in treatment of urinary incontinence, at the time of inserting the implant 8 into the puncture-through hole 500 so as to set the implant 8 indwelling there, the implant 8 is preliminarily housed in the medical tube 2 and is inserted into the puncture-through hole 500 together with the medical tube 2. Since the medical tube 2 is rigid at least at its curved section 231 as aforementioned, the medical tube 2 is prevented, even within the puncture-through hole 500, from being unwillingly deformed through being crushed by the biological tissue 700. Accordingly, the operation for insertion into the puncture-through hole 500 can be carried out easily and reliably.

In addition, in this procedure, after the implant 8 is inserted into the puncture-through hole 500 together with the medical tube 2, it is possible to separate the medical tube 2 and draw the medical tube 2 out of the puncture-through hole 500 easily. This ensures that the implant 8 can be reliably placed to indwell in the puncture-through hole 500.

In accordance with an exemplary embodiment, in this procedure, the operation as shown in FIG. 8, or the operation of pulling the guide wire 40 out, may be omitted.

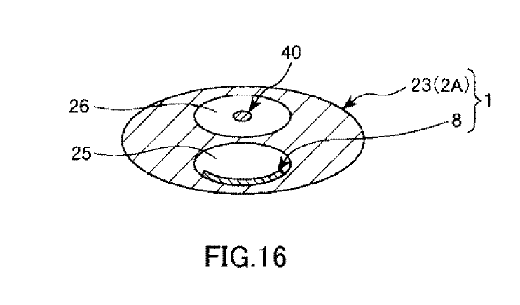

FIG. 16 is a cross-sectional view showing a second embodiment of the medical tube (medical tube assembly) of the present disclosure.

Hereafter, referring to this drawing, the second exemplary embodiment of the medical tube and the medical tube assembly according to the present disclosure will be described. The following description will center on differences from the aforementioned embodiment, and descriptions of the same items as above will be omitted.

The second exemplary embodiment can be the same as the first embodiment above, except for a difference in the number of lumens formed.

As shown in FIG. 16, a medical tube 2A in this embodiment is formed with a lumen 26 in which to insert the guide wire 40, in addition to a lumen 25 in which to insert the implant 8. The lumen 25 and the lumen 26 are formed independently from each other, which helps prevents interference between the implant 8 and the guide wire 40. Thus, for example, the insertion and pulling-out of the guide wire 40 can be performed relatively smoothly.

While the number of the lumens formed is two in this exemplary embodiment, this is not limitative. The number of the lumens 25, 26 may be a plurality, for example, three or more.

FIGS. 17 to 19 are sectional views for sequentially illustrating a method of using the medical tube (medical tube assembly) of the present disclosure in a third exemplary embodiment.

Hereafter, referring to FIGS. 17 to 19, the third exemplary embodiment of the medical tube and the medical tube assembly according to the present disclosure will be described. The following description will center on differences from the aforementioned embodiments, and descriptions of the same items as above will be omitted.

The third exemplary embodiment is the same as the first exemplary embodiment above, except that the separation part is omitted from the medical tube.

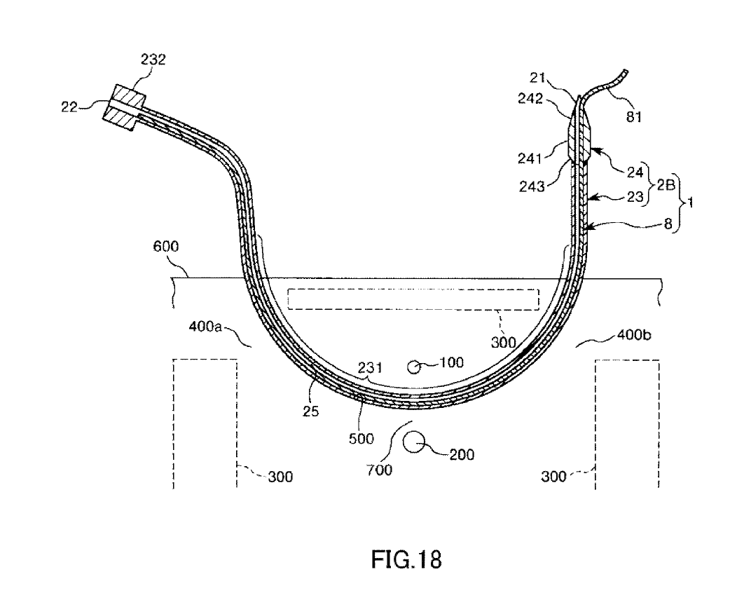

As shown in FIGS. 17 and 18, a medical tube 2B in this embodiment has a configuration in which such a separation part 235 as possessed by the medical tube 2 in the first exemplary embodiment above is omitted.

In addition, a distal portion 81 of the implant 8 is preliminarily set protruding from a distal end opening 21 of the medical tube 2B.

An example of the method of using the medical tube assembly 1 having the medical tube 2B configured as above will be described, referring to FIGS. 17 to 19. This using method and the using method in the first embodiment above are the same in regard of the operations as shown in FIGS. 1-6 and the operations as shown in FIGS. 10 and 11, and are different in regard of the operations as shown in FIGS. 7-9. Here, the different operations will be described.

After the operation as shown in FIG. 6, the medical tube assembly 1 in a state where the implant 8 is inserted in the medical tube 2B is prepared. As aforementioned, the distal portion 81 of the implant 8 is protruding from the distal end opening 21 of the medical tube 2B.

Then, in a state where a proximal portion of the guide wire 40 is inserted in the distal end opening 21 of the medical tube 2B, the medical tube assembly 1 is pushed distally relative to the guide wire 40, as shown in FIG. 17. This causes the medical tube assembly 1 to be inserted into and passed through the puncture-through hole 500, and brought into a state where its distal-side portion is protruding from the body surface 600 on the side of the obturator foramen 400b, whereas its proximal-side portion is protruding from the body surface 600 on the side of the obturator foramen 400a. Even in this state, the distal portion 81 of the implant 8 is protruding from the distal end opening 21 of the medical tube 2B.

Next, that portion of the medical tube assembly 1 which is protruding from the body surface 600 on the side of the obturator foramen 400b is grasped with one hand, and, keeping this condition, a proximal portion of the guide wire 40 is grasped with the other hand and pulled proximally. This results in that, as shown in FIG. 18, the guide wire 40 is drawn out of the medical tube assembly 1 (medical tube 2B), whereas the medical tube assembly 1 is left extending through the puncture-through hole 500. Even in this state, the distal portion 81 of the implant 8 can protrude from the distal end opening 21 of the medical tube 2B.

Subsequently, the distal portion 81 of the implant 8 is grasped with one hand, and, keeping this condition, a flange portion 232 of the medical tube 2B is grasped with the other hand and pulled proximally until the medical tube 2B is completely drawn out of the puncture-through hole 500. This results in that the implant 8 is left extending through the puncture-through hole 500.

Thereafter, the operations as shown in FIGS. 10 and 11 can be carried out sequentially.

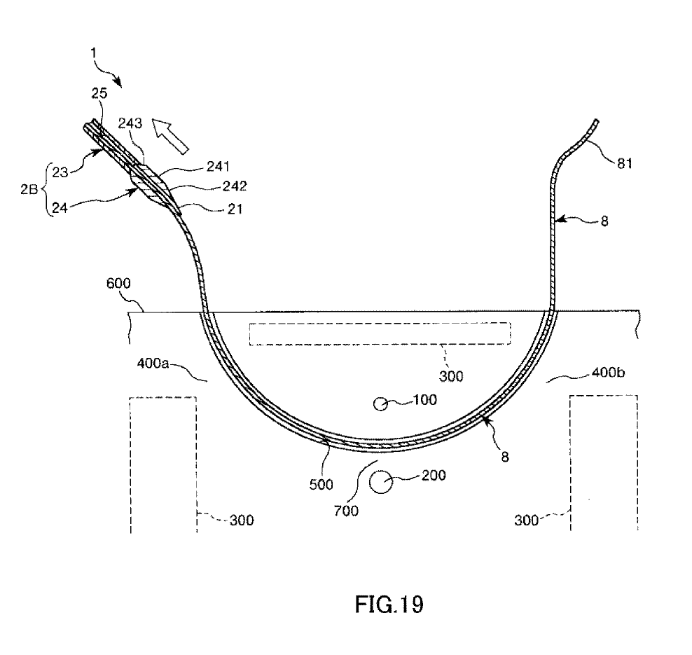

While the distal portion 81 of the implant 8 can be preliminarily positioned to protrude from the distal end opening 21 in the medical tube 2B, this is not restrictive. For example, the distal portion 81 of the implant 8 may be recessed from the distal end opening 21. In accordance with an exemplary embodiment, it can be preferable, for example, for the medical tube 2B to be so configured that the tube main body 23 and the head part 24 are separable from each other, which can help ensure that when the head part 24 is separated, the distal portion 81 of the implant 8 can protrude from the tube main body 23. With the head part 24 separated after the operation as shown in FIG. 18, the operation as shown in FIG. 19 can be reliably carried out.

While the medical tube and the medical tube assembly of the present disclosure have been described hereinabove with reference to the embodiments shown in the drawings, the disclosure is not limited to the embodiments. Each of the components of the medical tube and the medical tube assembly can be replaced with one having an arbitrary configuration that is able to exhibit an equivalent function. For example, an arbitrarily configured body or bodies may be added.

In addition, the medical tube and the medical tube assembly of the present disclosure may each be a combination of arbitrary two or more configurations (features) of the embodiments above.

While the curved section of the medical tube is so configured as to maintain the curved state in the circular arc shape by its own rigidity in each of the embodiments above, this configuration is not restrictive. For example, a configuration may be adopted wherein a rigid stylet having a curved portion curved in a circular arc shape or the like member is inserted in the curved section of the medical tube, so as to maintain the curved state.

In addition, the separation part of the medical tube may be composed of perforations.

The medical tube of the present disclosure is a medical tube in which an elongated implant is to be inserted, wherein the medical tube includes a tube having a distal end opening where a distal end of the distal end opening is open, and a proximal end opening where a proximal end of the proximal end opening is open, and the tube has a curved section where an intermediate portion in a longitudinal direction of the medical tube is curved and where a curved state is maintained. Therefore, when the medical tube is inserted into a living body, the curved section is prevented from being crushed (compressed) inside the living body. This can help ensure that, for example, in the case where an implant is preliminarily inserted in the medical tube, the operation of inserting the medical tube into a living body together with the implant can be carried out easily and reliably. In addition, after the inserting operation, it is required only to pull the medical tube out of the living body, whereby the implant is left as it is, so that the implant is easily and reliably placed to indwell in the living body.

Accordingly, the medical tube of the present disclosure has industrial applicability.

The detailed description above describes a medical tube and a medical tube assembly. The disclosure is not limited, however, to the precise embodiments and variations described. Various changes, modifications and equivalents can effected by one skilled in the art without departing from the spirit and scope of the disclosure as defined in the accompanying claims. It is expressly intended that all such changes, modifications and equivalents which fall within the scope of the claims are embraced by the claims.

What is claimed is:

1. A medical tube in which an elongated implant is to be inserted, the medical tube comprising:
a tube having a distal end opening where a distal end of the distal end opening is open, and a proximal end opening where a proximal end of the proximal end opening is open, the tube having a curved section where an intermediate portion in a longitudinal direction of the tube is curved and where a curved state is maintained.

2. The medical tube according to claim 1, wherein at least the curved section of the tube is rigid.

3. The medical tube according to claim 1, wherein the curved section is curved in a circular arc shape.

4. The medical tube according to claim 1, comprising:
a separation part where the tube is separable at the intermediate portion in the longitudinal direction of the tube.

5. The medical tube according to claim 4, wherein the separation part is disposed at a central portion in a longitudinal direction of the curved section.

6. The medical tube according to claim 5, comprising:
a marker for grasping of the central portion in the longitudinal direction of the curved section.

7. The medical tube according to claim 4,
wherein the tube is separated at the separation part into a first tube on a distal side and a second tube on a proximal side; and
the separation part having a fitting structure in which a distal portion of the second tube is fitted in a proximal portion of the first tube, before separation of the tube into the first tube and the second tube.

8. The medical tube according to claim 1, wherein at least a portion of the medical tube is circular in cross-sectional shape at the distal end opening.

9. The medical tube according to claim 8, comprising:
an enlarged diameter portion where the medical tube is enlarged in outside diameter at the distal end opening.

10. The medical tube according to claim 8, wherein at least a portion of the medical tube is flat in cross-sectional shape proximal to the distal end opening.

11. The medical tube according to claim 10, wherein a width of the flat shape is equal to or greater than a maximum outside diameter at the distal end opening of the medical tube.

12. The medical tube according to claim 10, wherein a thickness direction of the flat shape is oriented toward a center-of-curvature of the curved section.

13. The medical tube according to claim 1, comprising:
at least one lumen which opens respectively at the distal end opening and at the proximal end opening; and
an implant, which is inserted into the at least one lumen.

14. The medical tube according to claim 13,
wherein the at least one lumen is a plurality of the lumens; and
at least one guide wire, and wherein the at least one guide wire is inserted in each of the plurality of lumens other than the at least one lumen of the plurality of lumens in which the implant is inserted.

15. A medical tube assembly comprising:
a medical tube having a distal end opening where a distal end of the distal end opening is open, and a proximal end opening where a proximal end of the proximal end opening is open, the tube including a curved section where an intermediate portion in a longitudinal direction of the tube is curved and where a curved state is maintained; and
an elongated implant which is inserted in the medical tube.

16. The medical tube assembly according to claim 15, comprising:
a separation part where the medical tube is separable at the intermediate portion in the longitudinal direction of the medical tube, wherein the separation part is disposed at a central portion in a longitudinal direction of the curved section, and the tube is separated at the separation part into a first tube on a distal side and a second tube on a proximal side; and
a marker for grasping of the central portion in the longitudinal direction of the curved section.

17. A method of placing an implant in living body tissue, the method comprising:
forming a puncture hole by puncturing a living body tissue with a puncture apparatus, the puncture apparatus including a puncture needle and an outer tube;
withdrawing the puncture needle of the puncture member from the outer tube;
inserting a guide wire inserted into and passing through the outer tube, such that a distal portion of the guide wire protrudes from an opening on a distal end of the outer tube and a proximal portion of the guide wire protrudes from an opening on a proximal end of the outer tube;
withdrawing the outer tube from the puncture hole, while leaving the guide wire extending through the puncture hole;
inserting a medical tube assembly into the puncture hole, the medical tube assembly including a medical tube and an elongated implant within the medical tube, the medical tube having a distal end opening where a distal end of the distal end opening is open, and a proximal end opening where a proximal end of the proximal end opening is open, and wherein the medical tube includes a curved section where an intermediate portion in a longitudinal direction of the tube is curved and where a curved state is maintained;
pulling the guide wire out of the medical tube assembly; and
withdrawing the medical tube from the puncture hole and leaving the implant in a state in a state where a distal-side portion of the implant protrudes from a body surface on one side and a proximal-side portion of the implant protrudes from the body surface on another side.

18. The method according to claim 17, wherein the inserting the medical tube assembly with the implant into the puncture hole comprises:
inserting the guide wire in the distal end opening of the medical tube; and
distally pushing the medical tube assembly relative to the guide wire, which causes the medical tube assembly to be inserted into and passed through the puncture hole.

19. The method according to claim 18, wherein medical tube includes a separation part where the medical tube is separable at the intermediate portion in the longitudinal direction of the medical tube, wherein the separation part is located at a central portion in a longitudinal direction of the curved section, and wherein the medical tube is separated at the separation part into a first tube on a distal side and a second tube on a proximal side, and the separation part having a fitting structure in which a distal portion of the second tube is fitted in a proximal portion of the first tube, before separation of the tube into the first tube and the second tube, and a marker for grasping of the central portion in the longitudinal direction of the curved section;
causing the medical tube to be separated at the separation part into the first tube and the second tube; and
withdrawing the first and second tube from the puncture hole.

20. The method according to claim 19, comprising:
pulling a distal-side portion and a proximal-side portion of the implant to generate a tension on the implant; and
cutting away portions of the implant which are not necessary.
